(19) United States
(12) Patent Application Publication
(10) Pub. No.: US 2026/0198625 A1
(43) Pub. Date: Jul. 16, 2026

(54) MASKS, HELMETS AND COMPONENTS THEREOF

(71) Applicant: Warren Blake, Rotorua (NZ)

(72) Inventor: Warren Blake, Rotorua (NZ)

(21) Appl. No.: 19/137,449

(22) PCT Filed: Apr. 28, 2023
(86) PCT No.: PCT/NZ2023/050047

§ 371 Date: Jun. 10, 2025

(30) Foreign Application Priority Data
Dec. 15, 2022 (AU) 2022903854

(51) Int. Cl. A41D 13/11; A42B 3/22; A61M 16/06; A62B 18/00; A62B 18/02; A62B 18/08

(52) U.S. Cl. CPC A41D 13/1161; A41D 13/1184; A42B 3/225; A61M 16/0683; A62B 18/003; A62B 18/02; A62B 18/08

(57) ABSTRACT

Disclosed are mask systems which are configured with a front portion and a rear portion, wherein the front portion is connected to the rear portion by a hinge mechanism which allows both for pivoting of the front portion with respect to the rear portion, as well as translational movement of the front portion relative to the rear portion. In some examples, the hinge mechanism may slide into a channel in order to prevent pivoting of the front portion with respect to the rear portion in use.

### STATEMENT OF CORRESPONDING APPLICATIONS

This application is based on the provisional specification filed in relation to Australian Patent Application No. 2022903854, the entire contents of which are incorporated herein by reference.

### TECHNICAL FIELD

The present disclosure relates to mask helmets and components thereof. The technology may find particular application as a workshop mask or helmet, such as a welding or grinding mask, however this should not be seen as limiting on the scope of the technology.

### BACKGROUND

Most workshop masks use a simple mounting system which includes a fastening band, which is secured around the circumference of the user's head, and includes a two-point pivot for a face shield. This configuration allows for the face shield to be pivoted up, out of the way as required.

Some examples of these types of masks include one or more support straps which sit across the top, and/or crown of the user's head in use. These support straps can help to support the weight of the mask without requiring excessive tension around the circumference of the user's head.

Such masks, in order to be comfortable and secure, need to be adjusted and customised for each user. For example, to account for different head shapes and sizes, it can be beneficial for the straps and headband to be adjusted on a person-by-person basis for the specific head dimensions of each user. Accordingly, these masks can be cumbersome in situations where two or more people need to use the mask.

A further issue with two-point pivot masks is that the weight of the mask may not be evenly distributed across the head of the user, with the majority of the weight coming from the face shield, which is attached to the front of the user's head. This can cause issues such as muscle fatigue with prolonged use. In addition, the rear tightening mechanisms used in these masks make it uncomfortable for a user to work in the supine position, i.e., lying on their back.

In some applications, it can be desirable for the user to wear a dust mask, air filter, or full respirator while also wearing a protective mask, such as a welding or grinding mask. With existing masks, this typically means that the user is restricted to using low-profile dust masks, filters and respirators, each with their own mounting systems that can interfere with the mounting systems of the protective mask. This configuration also adds to the time taken to put on and remove the mask systems, adds cost and weight, all of which makes the existing systems cumbersome.

Aspects of the technology of the present disclosure are directed to overcoming one or more of the problems discussed above. It is an object of the present invention to address one or more of the foregoing problems or at least to provide the public with a useful choice.

Further aspects and advantages of the present disclosure will become apparent from the ensuing description which is given by way of example only.

### SUMMARY

According a first aspect of the present technology, there are provided a mask system and components thereof.

According to another aspect of the present technology, there is provided an improved workshop mask.

According to another aspect of the present technology, there is provided a mask system comprising:
a front portion configured to engage with the front of a user's head in use;
a rear portion configured to engage with the rear of the user's head in use;
a hinge mechanism which connects the front portion to the rear portion; and
   wherein, in use, the hinge mechanism is configured to move between a first position, in which the hinge mechanism allows for pivotal movement of one of the front portion and rear portion relative to the other of the front portion and rear portion, and a second position, in which the hinge mechanism does not allow for pivotal movement of one of the front portion and rear portion relative to the other of the front portion and rear portion.

According to another aspect of the present technology, there is provided a mask system comprising:
a front portion configured to engage with the front of a user's head in use; and
a rear portion configured to engage with the rear of the user's head in use;
   wherein one of the front portion and the rear portion comprise a hinge mechanism, configured to move within a channel provided to the other of the front portion and the rear portion, to allow the front portion to slide with respect to the rear portion, and
   wherein the hinge mechanism comprises a joint, and when the joint is withdrawn from the channel, the hinge mechanism is able to pivot around the joint to allow the one of the front portion and rear portion to pivot relative to the other of the front portion and rear portion.

According to another aspect of the present technology, there is provided a method of using a mask system substantially as described above, wherein the method comprises the steps of:
A user putting the mask system on their head and operating the hinge mechanism; or
A user operating the hinge mechanism and removing the mask system from their head.

According to another aspect of the present technology, there is provided a method of putting on a mask system comprising a front portion and a rear portion, wherein the method comprises the steps of:
Slidably moving of one of the front portion and rear portion relative to the other of the front portion and rear portion so as to remove a first hinge component of a hinge mechanism from a channel;
Pivoting one of the front portion and rear portion relative to the other of the front portion and rear portion to increase the separation between the front portion and the rear portion;
Positioning the mask system over the head of the user;
Pivoting one of the front portion and rear portion relative to the other of the front portion and rear portion to decrease the separation between the front portion and the rear portion;
Sliding one of the front portion and rear portion relative to the other of the front portion and rear portion to insert the first hinge component into the channel.
According to another aspect of the present technology, there is a method of putting on a mask system as described above, further comprising the step of attaching a securing mechanism between the front portion and the rear portion to prevent the front portion and rear portion sliding relative to each other.

According to another aspect of the present technology, there is provided a method of removing a mask system comprising the steps of:
Removing a securing mechanism between a front portion and a rear portion of the mask system to allow the front portion and rear portion to slide relative to each other;
Sliding one of the front portion and rear portion relative to the other of the front portion and rear portion to remove a first hinge component of a hinge mechanism from a channel;
Pivoting one of the front portion and rear portion relative to the other of the front portion and rear portion to decrease the separation between the front portion and the rear portion;
Removing the mask system from the head of a user;

According to another aspect of the present technology, there is a method of removing a mask system as described above, further comprising the step of:
pivoting one of the front portion and rear portion relative to the other of the front portion and rear portion to decrease the separation between the front portion and the rear portion; and sliding one of the front portion and rear portion relative to the other of the front portion and rear portion to remove the first hinge component of the hinge mechanism from the channel.

In examples, the front portion may comprise any one or more of: a visor, a breathing filter, a face engaging seal, a face shield and/or an air conduit.

In examples, the front portion and/or visor may comprise any one or more of Ultraviolet (UV) protection, auto darkening technology, a polarising filter, air vents, and/or a visor defogging feature.

In examples, the front portion may comprise side portions which extend rearwardly, or otherwise in a substantially posterior direction from the front portion.

In examples, the front portion and the rear portion may together provide a full head helmet.

In examples, the rear portion may include side portions, which extend forwardly or otherwise in a substantially anterior direction from the rear portion.

In examples, the front portion may comprise a forehead portion configured to engage with the forehead of the user.

In examples, the forehead portion may comprise a forehead contacting portion.

In examples, the forehead contacting portion may comprise a resiliently deformable material configured to deform to the shape of the user's head in use.

In examples, the resiliently deformable material may comprise a fabric, an elastomer, a foam or a polymer.

In examples, the forehead contacting portion may be attached to the front portion via an adjusting means.

In examples, the adjusting means may be configured to allow the positioning of the forehead contacting portion to be adjusted relative to the front portion.

In examples, the adjusting means may comprise a threaded shaft and a complementary threaded aperture. For example, rotation of the threaded shaft within the threaded aperture may be configured to adjust the position of the forehead contacting portion relative to the front portion, for example in an anterior-posterior direction or a superior-inferior direction relative to the user's head.

In examples, the threaded shaft may be provided with an adjustment known, which the user can rotate in use to actuate the adjusting means.

In examples, the rear portion may comprise a crown and/or nape engaging portion configured to engage with the crown and/or nape of the user.

In examples, the crown and/or nape engaging portion may comprise an adjustment mechanism configured to allow the positioning of the crown and/or nape engaging portion to be adjusted relative to the rear portion.

In examples, the crown and/or nape engaging portion may comprise a resiliently deformable material. For example, the resiliently deformable material may comprise a fabric, an elastomer, a foam or a polymer.

In examples, the rear portion may comprise ear protection, such as earmuffs.

In examples, the ear protection may be provided with an adjustment mechanism such that the positioning of the ear protection may be adjusted relative to the rear portion.

In examples, the rear portion may comprise a substantially planar rear surface.

In examples, the hinge mechanism may comprise a first hinge component and a second hinge component, the first hinge component being rotatably connected to the second hinge component.

In examples the first component and the second component may be constructed of a rigid material such as a metal, polymer, or ceramic.

In examples, the first hinge component may be rotatably connected to the second hinge component using a joint, such as a flexible joint.

In examples, the hinge mechanism may be fixedly attached to one or more of the front portion and the rear portion, and the other of the front portion and rear portion may comprise a channel configured to receive the hinge mechanism.

In examples, the hinge mechanism may be configured to act as a protrusion which in use slides within the channel, so as to provide sliding movement between the front portion and the rear portion.

In examples, the hinge mechanism may have a substantially arcuate profile when viewed from a side.

In examples, the channel may be configured to provide a substantially arcuate path through which the hinge mechanism can slide in use.

In examples, the hinge mechanism may be attached to a stop surface of the rear portion using one or more of a fastener, adhesive, moulding, heat-staking, or welding process. In alternative examples, the hinge mechanism may be formed integrally with the rear portion, for example by way of a moulding process.

In examples, the hinge mechanism may comprise a flange extending outwardly from one or more sides of the hinge mechanism to provide a channel through which the front or rear portion may slide in use.

In examples, the mask system may further comprise a first retention mechanism configured to maintain the hinge mechanism in sliding engagement with one or more of the front portion and/or rear portion. For example, the first retention mechanism may comprise at least one protrusion which in use engages with one or more stops to prevent or otherwise limit removal of the hinge mechanism.

In examples, the mask system may further comprise a second retention mechanism configured to secure the front portion at a substantially fixed position with respect to the rear portion.

In examples, the second retention mechanism may comprise a fastener such as a hook-and-loop fastener, a clamp, buttons, domes, hooks, buckles, and/or a latch.

The above and other features will become apparent from the following description and the associated drawings.

### BRIEF DESCRIPTION OF THE DRAWINGS

Further aspects of the present disclosure will become apparent from the following description which is given by way of example only and with reference to the accompanying drawings in which:

FIG. 1A is a side view of one example of a mask system in accordance with the present technology.

FIG. 1B is a side view of a further of a mask system in accordance with the present technology.

FIG. 1C is a side view of a further of a mask system in accordance with the present technology.

FIG. 1D is an underside view of the mask system of FIG. 1C.

FIG. 1E is a close-up view of an adjustment mechanism of a mask system in a first position.

FIG. 1F is a close-up view of the adjustment mechanism of FIG. 1E in a second position.

FIG. 2 is a side view of the mask system of FIG. 1C with the front portion and rear portion pivoted relative to one another.

FIG. 3A is a top view of a hinge mechanism in accordance with the present technology.

FIG. 3B is a side view of the hinge mechanism of FIG. 3A.

FIG. 3C is a side view of the mask system of FIG. 1C with the front and rear portions separated. The internal channel has also been illustrated for convenience.

FIG. 3D is a perspective view of a retention mechanism in accordance with one example of the present technology.

FIG. 3E is a side view of the mask system of FIG. 1C with the front and rear portions mating. The internal channel has also been illustrated for convenience.

FIG. 4 is a top view of a front portion of a mask system in accordance with the present technology.

FIG. 5A is a top view of a mask system, with a retention mechanism securing the front portion to the rear portion, in accordance with one example of the present technology.

FIG. 5B is a top view of the mask system of FIG. 5A with the retention mechanism disengaged.

FIG. 5C is a rear view of the mask system of FIG. 5A, with the retention mechanism securing the front portion to the rear portion.

FIG. 5D is a rear view of the mask system of FIG. 5A, with the retention mechanism disengaged.

FIG. 5E shows a front view of the mask system of FIG. 5A.

FIG. 6A shows another example of a mask system according to the present technology.

FIG. 6B shows another example of a mask system according to the present technology.

FIG. 6C shows another example of a mask system according to the present technology.

FIG. 7A shows a perspective view of a rear portion according to the present technology.

FIG. 7B shows a side view of the rear portion of FIG. 7A.

FIG. 7C shows an exploded end view of the rear portion of FIG. 7A with a hinge mechanism according to the present technology.

FIG. 7D shows an exploded perspective view of FIG. 7C.

FIG. 7E shows a partial end view of a channel according to the present technology.

FIG. 7F shows a top-down view of a front portion with the channel outline shown.

FIG. 7G shows a top-down view of a rear portion according to the present technology.

FIG. 7H shows a top-down view of the front portion of FIG. 7F engaged with the rear portion of FIG. 7G.

FIG. 7I shows a partial end view of the front and rear portions of FIG. 7H.

FIG. 7J shows a top-down view of a rear portion with a hinge mechanism attached thereto according to the present technology.

FIG. 7K shows a partial end view of the rear portion and hinge mechanism of FIG. 7J.

FIG. 7L shows an assembled partial end view of a mask system with a retention mechanism in an open state according to the present technology.

FIG. 7M shows an assembled partial end view of a mask system with a retention mechanism in a closed state according to the present technology.

### DETAILED DESCRIPTION

### Mask System Overview

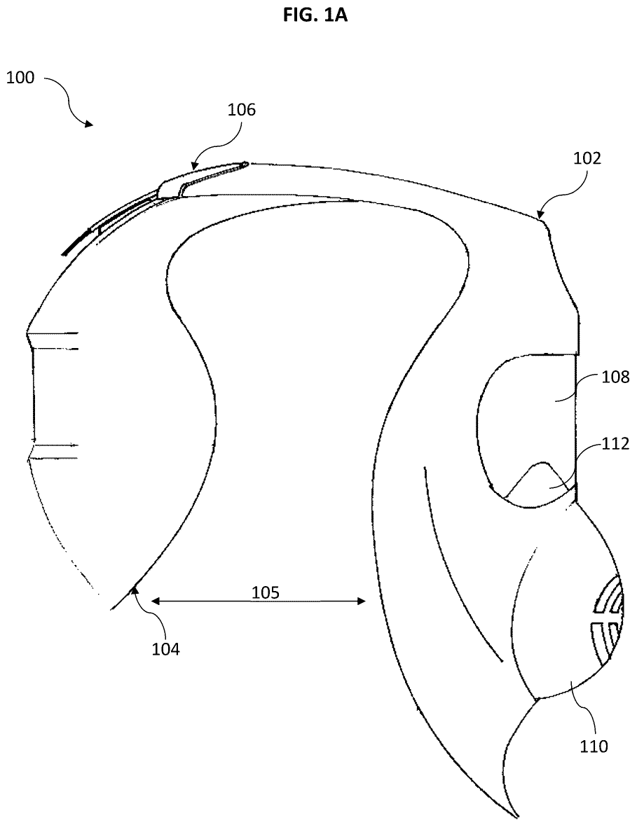

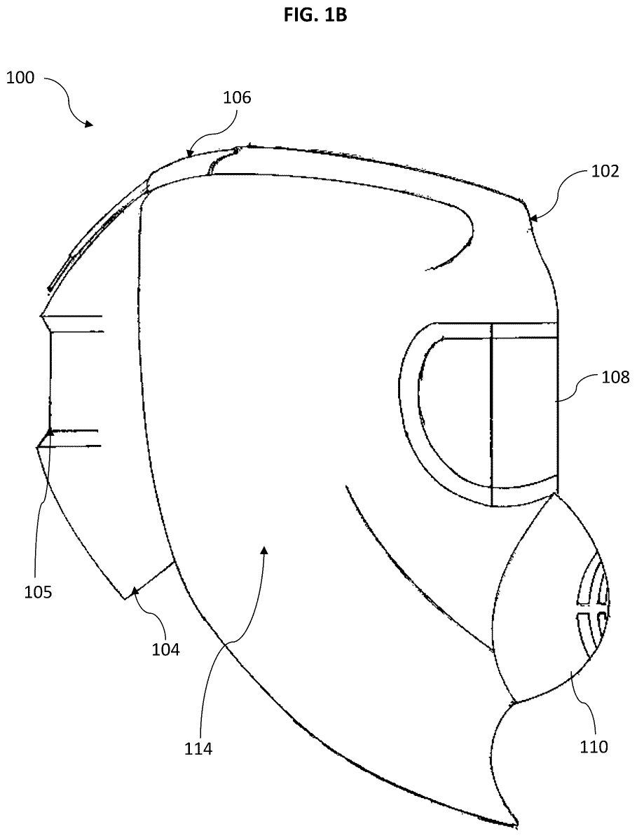

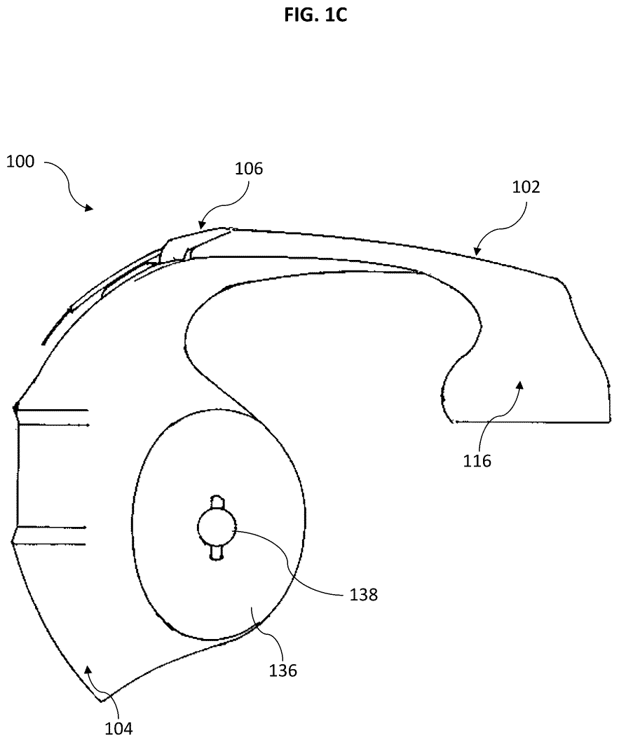

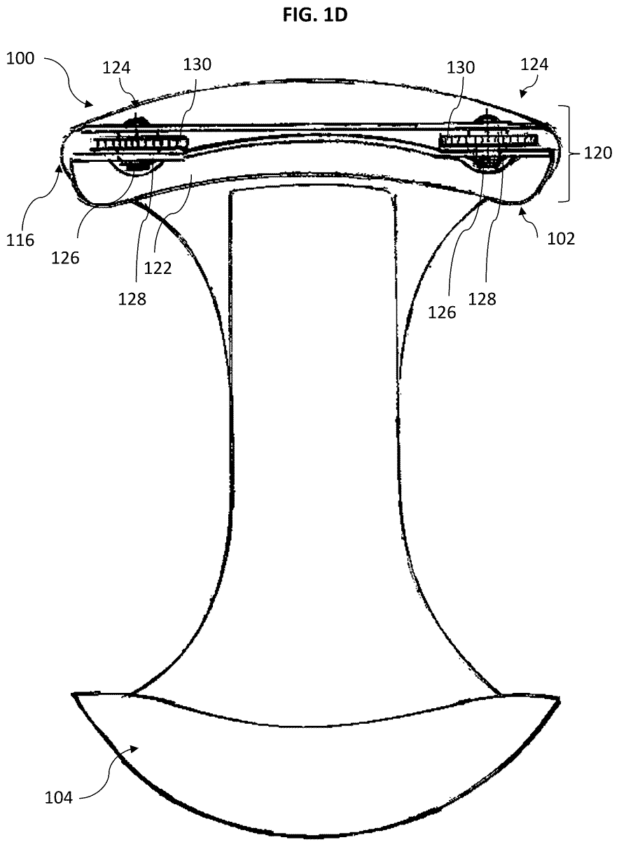

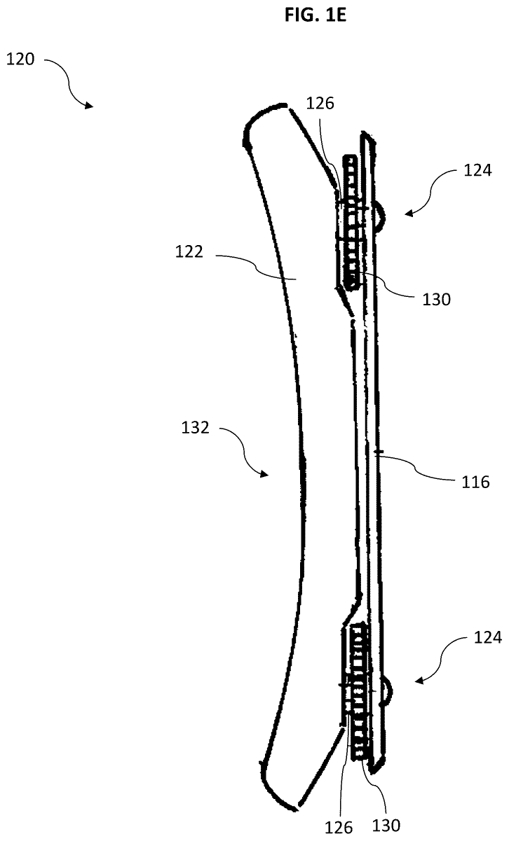

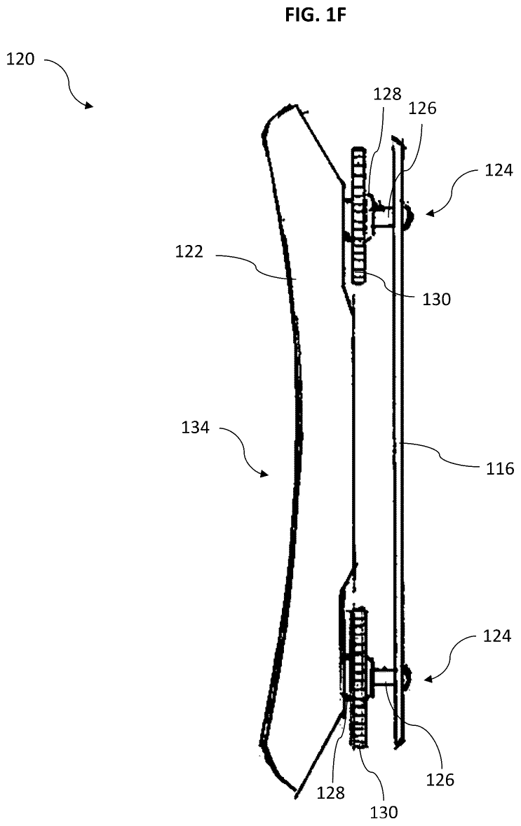

FIG. 1A shows a first example of a mask system 100 in accordance with one example of the present technology. In this example the mask system 100 comprises a front portion 102 and a rear portion 104. Connecting the front 102 and rear portions is a hinge mechanism 106 which allows the front portion 102 and the rear portion 104 to pivot or otherwise rotate with respect to each other. The hinge mechanism is also configured such that the front portion and the rear portions are slideable relative to each other, which is particularly advantageous as will be apparent from the ensuing description in which each of the foregoing components of the mask are described herein in greater detail. Although reference is generally made to masks and mask systems throughout the present specification, it should be appreciated that the principles of the present technology may be applied to protective headgear such as helmets or the like, and examples of this are also described herein.

The present technology allows for many mask configurations, and one way of accommodating different mask requirements is to provide variations to the front portion 102 of the mask system 100.

In the illustrated example of FIG. 1B, the front portion comprises a visor 108, and a breathing filter 110, which in this example includes a face engaging seal 112, configured to form a seal with or around one or more of the airways of the patient. For example, the face engaging seal 112, may be configured to seal around the nose and/or mouth of the user in use to ensure that the air breathed by the user passes through the filter first.

The visor 108 can similarly be configured for any suitable application. For example, in some applications the visor 108 may simply provide the user with eye protection, while in other examples the visor may include any one or more of:
UV protection.
Auto darkening technology, which should be familiar to those skilled in the art; for example, when used in welding applications.
Light polarisation, i.e., a polarising filter.
A visor defogging feature, such as air vents configured to pass air across the inside surface of the visor.

In some examples of the technology, such as the example shown in FIG. 1B, the front portion 102 includes side portions 114, which extend rearwardly, or otherwise in a substantially anterior-posterior direction from the front portion 102. In the illustrated example, the side portions 114 are configured to cover the sides of the user's head to substantially provide a full head helmet, when assembled together with the rear portion 104.

It should be appreciated that in some forms of the technology, the rear portion 104 may include the side portions 114 which extend along the sides of the user's head in use. For example the side portions may extend in an posterior-anterior direction along the side of the user's ear to provide a full head helmet. In other words, the front portion 102 and rear portion 104, together co-operate to provide side of head protection, and/or a full head helmet.

In some examples of the technology, such as the example shown in FIG. 1C, the front portion 102, may be configured to facilitate mounting to the head of the user. For example, the front portion 102, may comprise a forehead portion 116, configured to engage with the forehead of the user, in use.

It should further be appreciated that while in the foregoing examples the front portion 102, is configured to provide utility in the form of a visor or breathing filter, these components are optional, and as illustrated in FIG. 1C, may be omitted entirely. In other examples, the present technology may provide a modular mask system in which components of the technology may be releasably attached to the front 102 or rear portions 104. For example, the visor 108, breathing filter 110, and/or face engaging seal 112 may be configured to releasably attach to the forehead portion, for example using any fastening system known to those skilled in the art, including screws, bolts, clips, and clamps.

In some examples of the technology, the forehead portion 116, may be configured to engage directly with the forehead of a user in use, for example the forehead portion 116 may comprise a forehead contacting portion 122, which is fixedly, or releasably attached to the forehead portion 116, such as a cushion or other suitable resiliently deformable component or material which can comfortably conform to the shape of the user's head. In some examples the cushion may comprise materials such as a fabric, an elastomer, a foam or a polymer. In other examples of the technology, the forehead contacting portion may be integral to the forehead portion 116, for example the forehead contacting portion may be provided by the underside or internal surface of the forehead portion 116.

In other examples, of the technology, the forehead portion 116 may comprise an adjustment mechanism 120, configured to allow for adjustment of the forehead portion in one or more directions with respect to the forehead of the user. For example, FIG. 1D shows an underside view of the example mask of FIG. 1C.

In this example the adjustment mechanism 120 comprises a forehead contacting portion 122, configured to engage with the forehead of the user in use. The forehead contacting portion 122 is attached to an adjusting means 124 for example the adjusting means 124 shown is provided by such as a threaded shaft 126 and complementary threaded aperture 128 such that rotation of the threaded shaft 126 causes the shaft to move within the threaded aperture 128 to move the forehead contacting portion 122 relative to the forehead portion 116.

To enable easy rotation of the threaded shaft 126, an adjustment knob 130 is provided which the user can rotate, for example using their fingers to actuate the adjustment mechanism 120 to move the forehead contacting portion relative to the forehead portion, such as in an anterior-posterior direction relative to the user's head.

Accordingly, with reference to FIGS. 1E and 1F, the adjustment mechanism 120 is configured to provide positional adjustment of the forehead contacting portion 122, between a first position 132 wherein the forehead contacting portion 122 is closer to the forehead portion 116 of the mask system 100, as shown in FIG. 1E and a second position 134 wherein the forehead contacting portion 122, is further from the forehead portion 116 than in the first position, as shown in FIG. 1F, including any position therebetween.

In the illustrated examples the adjustment mechanism 120 comprises two sets of adjusting means 124, however this should not be seen as limiting on the technology, and any number of adjusting means 124 may be provided including a single adjusting means 124, or more than two adjusting means 124. Similarly, while the adjusting means 124 shown are configured to adjust the position of the forehead contacting portion 122 in a substantially anterior-posterior direction relative to the head of the user, this should not be seen as liming. For example, the adjusting means 124 may be configured to move the forehead contacting portion 122 in any direction. In some examples a first adjusting means 124, may be configured to move the forehead contacting portion in a substantially anterior-posterior direction relative to the head of the user, while a second adjusting means 124, may be configured to move the forehead contacting portion in a substantially superior-inferior direction relative to the head of the user.

It should be appreciated that the adjustment mechanism 120 has been described in relation to the example of FIC. 1C, however this may be provided to any example of a mask system 100 as described herein, for example including components such as visors 108, breathing filters 110, and face engaging seals 112.

### Rear Portion

The rear portion 104 is configured to engage with the rear of a user's head and may include an adjustment mechanism 120 as described in relation to the front portion 102, for example to adjust the positioning of a crown or nape engaging portion (not shown) with the crown or nape of a user's head. For example, the crown or nape engaging portion may comprise a user-contacting portion constructed from a resiliently deformable material, such as an elastomeric, fabric or cushion liner to provide increased comfort to the user.

The rear portion 104 has a domed shape which is generally configured to match the shape of the crown of a user's head. Accordingly, while most adults can be accommodated with a limited range of different rear portion sizes and configurations, it may be desirable to include a smaller rear portion 104 when the mask system 100 is configured for use with children, and/or a deeper nape region when configured for female use. One way of achieving multiple shapes is to provide removable material inserts, for example the resiliently deformable material may be removed and replaced with a differently dimensioned section of resiliently deformable material.

The rear portion 104, may include optional features such as ear/hearing protectors 136, in the form of integrated earmuffs, as shown in FIG. 1C. In some examples the ear protectors may further include speakers.

The ear protectors 136 may also include an adjustment mechanism 138 configured to allow the positioning of the ear protectors to be moved relative to the rear portion 104, such as in an inferior-superior direction relative to the user's head and/or a posterior-anterior direction.

The rear portion 104 described herein has a substantially planar rear surface 105 which, unlike existing mask systems which use a band tightening knob at the rear of the mask system, allows the present mask system to be used when the user is lying on their back, i.e., in the supine position, without user discomfort.

### Hinge Mechanism

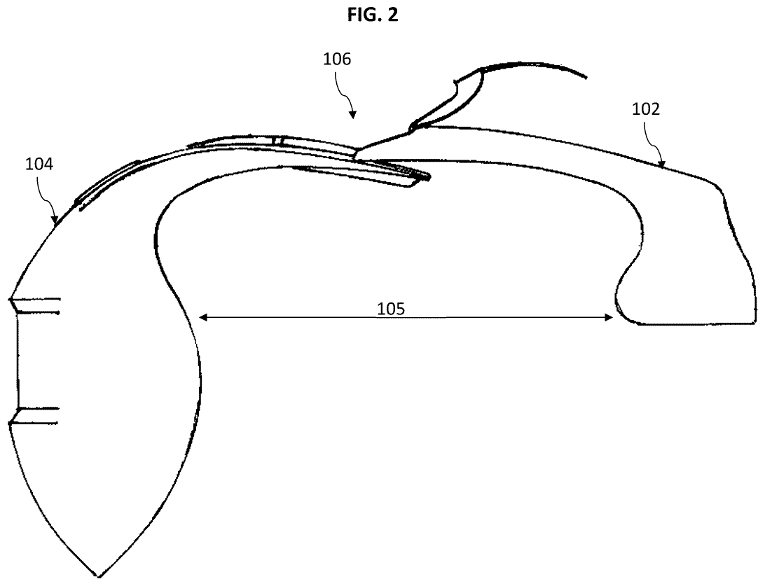

The hinge mechanism 106 connects the front portion 102 to the rear portion 104, such that the front portion 102 can pivot relative to the rear portion (or vice versa) during adjustment of the mask system 100. FIG. 2 shows one example of the mask system of the present technology in an open position in which the front portion 102 and rear portion 104 are pivoted with respect to one another about the hinge mechanism 106. Accordingly, the pivoting of the front portion relative to the second portion allows a quick and easy way for the opening 105 to be increased to facilitate positioning the mask system on the head of a user, and/or removing the mask system from the head of the user.

To provide the pivoting action, the hinge mechanism 106 may comprise any joint 306 known to those skilled in the art, including but not limited to, a hinge, a spring joint, a living hinge, and the use of a flexible material. It should also be appreciated that the pivoting action described herein does not need to occur around a single pivot point, but instead can comprise a plurality of pivot points, or otherwise, not have a defined pivot point, such as by using a resiliently deformable hinge material.

Once the mask is on position on the user's head it can be advantageous for the relative pivoting of the front portion 102 to the rear portion 104 to be limited. Accordingly, systems are described herein for allowing the hinge mechanism to move between a first position in which the hinge mechanism allows for the rear portion to be pivoted relative to the front portion, and a second position, in which the hinge mechanism does not allow the rear portion to be pivoted relative to the front portion.

In addition to pivoting, it can be desirable for the mask system 100 to allow the front portion 102 to translate or otherwise move relative to the rear portion 104. In other words, by allowing the front portion to move towards the rear portion, the front and rear portions may engage with the front and rear of the user's head, irrespective of the size of the user's head. Accordingly, the hinge mechanism 106 of the present technology is also configured to allow sliding movement of the front portion 102, towards and away from the rear portion 104 and vice versa. As such the present technology may accommodate a range of head sizes.

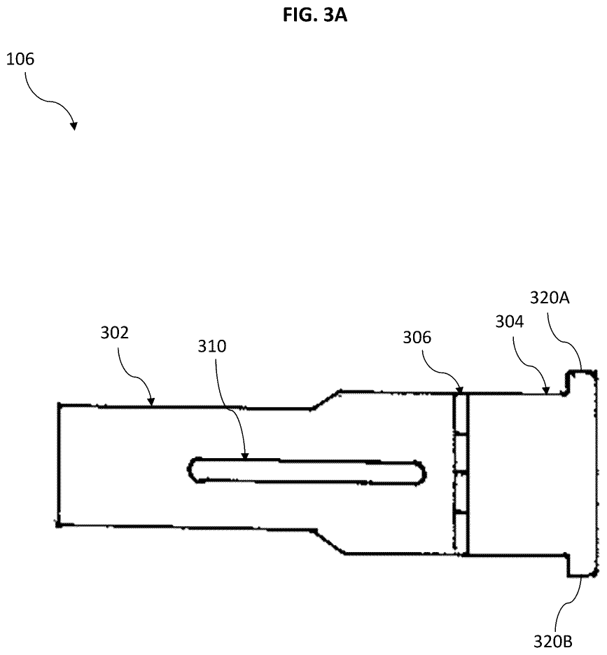

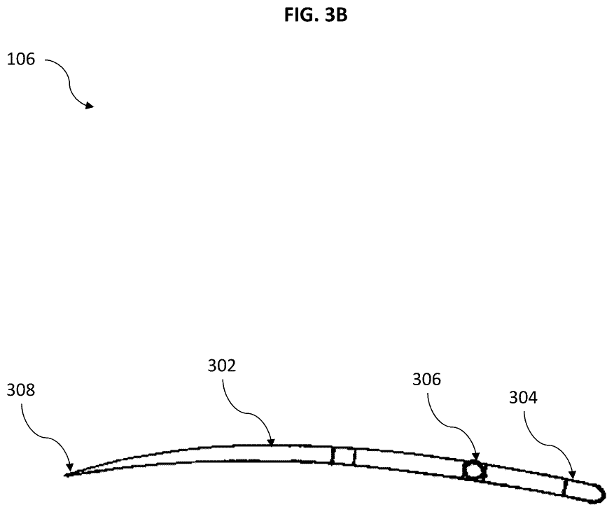

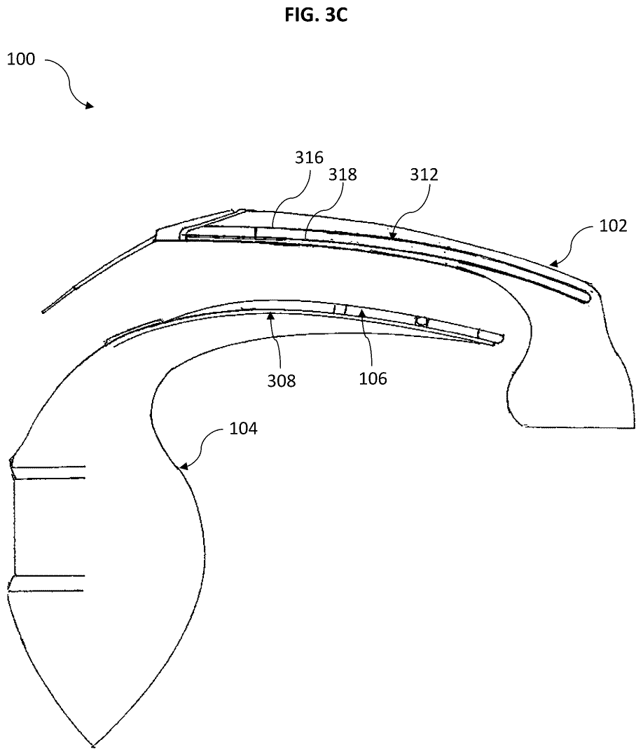

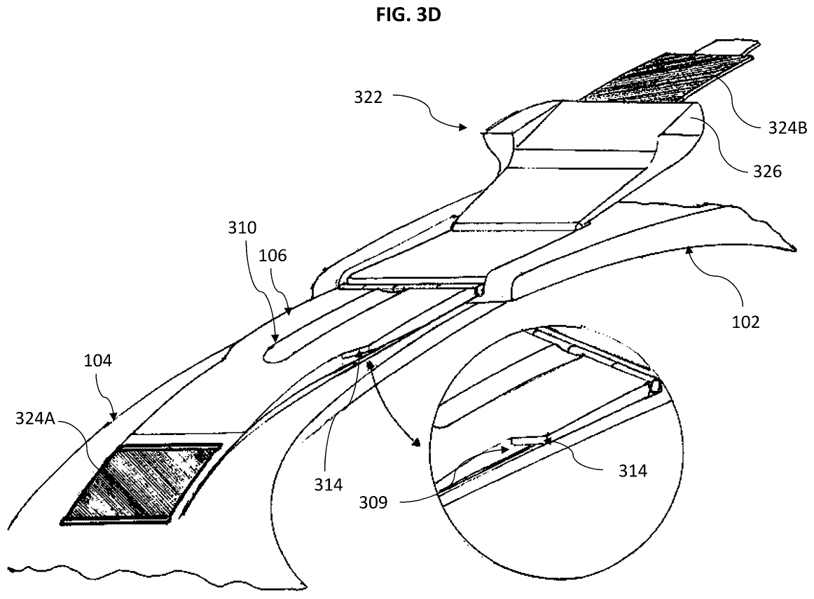

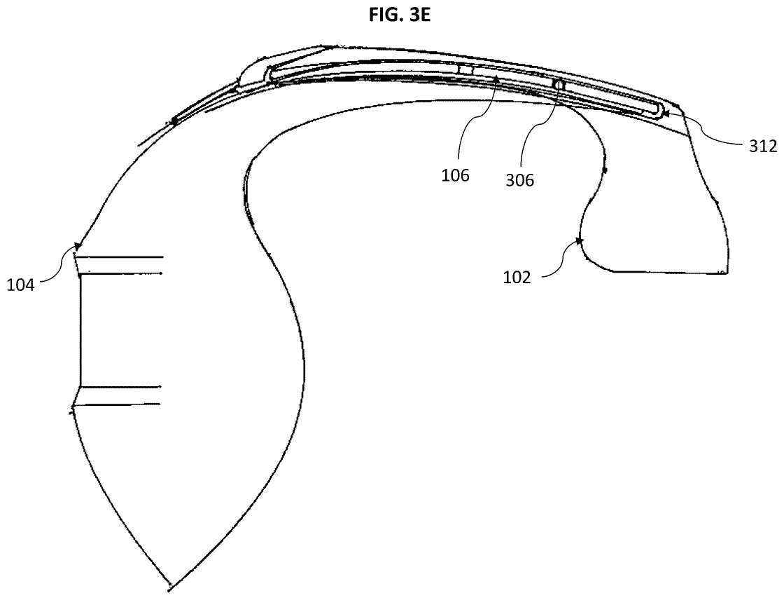

Referring to FIGS. 3A and 3B, one example of a hinge mechanism 106 is shown in accordance with the present technology. In this example the hinge mechanism 106 comprises a first hinge component 302, and a second hinge component 304, the hinge components (which may also be described as halves) 302, 304 each being connected by a joint 306 to provide a hinge mechanism.

In the examples shown the hinge halves 302, 304 may be constructed of a rigid material such as steel, however any suitable rigid material may be used including polymers, metals, and ceramics. Referring the to the side view of FIG. 3B, both hinge halves 302, 304 have a curved profile. The distal end 308 of the first hinge component 302 is shown as tapering to a point primarily for aesthetic reasons, and this should not be seen as a limiting feature of the technology.

In some examples of the technology, the hinge mechanism 106 may further comprise a guide 310, which may include a hollowed channel, recess, aperture or protrusion in the hinge mechanism 106, which is configured to, in use, engage with a complementary structure 310B on the rear portion in the channel so as to assist with guiding the sliding movement of the hinge mechanism through the channel while minimising any yawing motion. For example, the guide 310 may reduce the forces which would otherwise be imparted to the walls of the channel as the hinge mechanism slides through the channel. This can reduce occurrences of racking, or binding of the hinge mechanism within the channel.

FIG. 3C shows a side view of the respective front 102 and rear portions 104, with the hinge mechanism 106 secured to the top surface 308 of the rear portion 104. The hinge mechanism may be secured to the top surface 308 of the rear portion 104, using any methods known to those skilled in the art including using one or more fasteners, adhesives, or via a moulding, heat-staking, or welding process. For example, one or more fasteners may be inserted through an aperture in the top surface of the hinge mechanism 106. The foregoing example should not be seen to be limiting on the technology, for example the hinge mechanism may be configured to attach to any surface of the rear portion, or the front portion, including being formed integrally with the front portion, or the rear portion. The hinge mechanism simply needs to provide a protrusion which can be slidably engaged with a channel 312 in the corresponding front/rear portion.

In use the hinge mechanism 106 slides into a channel 312 within the front portion 102 of the mask system 100. The channel 312 is shown as being an internal channel in the front portion, however this should not be seen as limiting on the technology, and for example the channel 312 may instead be provided on the top or bottom surface of the front portion 102.

For the hinge mechanism 106 to slide into the channel 312, the sides of the hinge mechanism 106 need to be elevated above the top surface 308 of the rear portion 104. One method of achieving this is to fasten or otherwise secure the hinge mechanism onto a raised section 702 of the rear portion 104, such that a track remains under the sides of the hinge mechanism. Alternatively, or additionally, the hinge mechanism may be provided with a flange 314 (as shown in FIG. 3D) which forms a track 309 between the top surface 308 of the rear portion and the hinge mechanism 106, through which the front portion 102 can slide in use. This channel provides a guide for the front portion to slide through in use, i.e., with respect to the rear portion, and can further help to prevent yawing, or pitching motion of the hinge mechanism and associated front and rear portions in use. These features in combination may allow the present technology to provide a mask system which is comfortable, secure, easy to use and stable on the user's head.

Referring to FIG. 3C, it can be seen that the channel has a curvature which substantially corresponds to the curvature of the hinged mechanism as illustrated in FIG. 3B. In this way, the front and rear portions must be moved in an arcuate path to be slidably engaged with one another. This also means that, when the mask system 100 is assembled on a user's head, the forces imparted to the front portion 102 by the user's forehead (and in the case of respiratory masks, potentially the face engaging seal 112) and the rear portion 104 by the rear of the user's head are directly opposite to one another, and therefore do not encourage the two halves of the mask system to separate. Rather to separate the front portion from the rear portion, the corresponding halves need to be encouraged to follow the arcuate path provided by the hinge mechanism and the channel.

In some examples of the technology, it can be advantageous for the centre or apex of the curvature of the hinged mechanism to be positioned forwardly of the apex of the user's head in use. In this way, the forces imparted to the mask system 100 by engagement with the front and rear of the user's head are less likely to be aligned with the arcuate path of the hinge mechanism and channel, thereby further resisting the front portion slidably disengaging from the rear portion.

In practice, to separate the opposing halves of the mask system, a user generally needs to hold the rear portion with one and, and with the other hand, draw the front portion forwards and downwards, following an arcuate path corresponding to the shape of the channel.

While sliding the hinge mechanism 106 along the channel allows for the relative positioning of the front and rear portions to be adjusted, another feature of the present technology is the ability, to pivot the two halves apart for easy donning and removal of the mask system. Accordingly, when the two halves are sufficiently separated, the joint 306 is positioned outside of the channel, the rear portion is able to pivot relative to the front portion. Conversely, when the two halves are sufficiently assembled (as shown in FIG. 3E), the joint 306 is positioned within the channel, and supported by the top 316 and bottom 318 walls of the channel 312, substantially preventing any pivotal motion.

### Retention Mechanisms

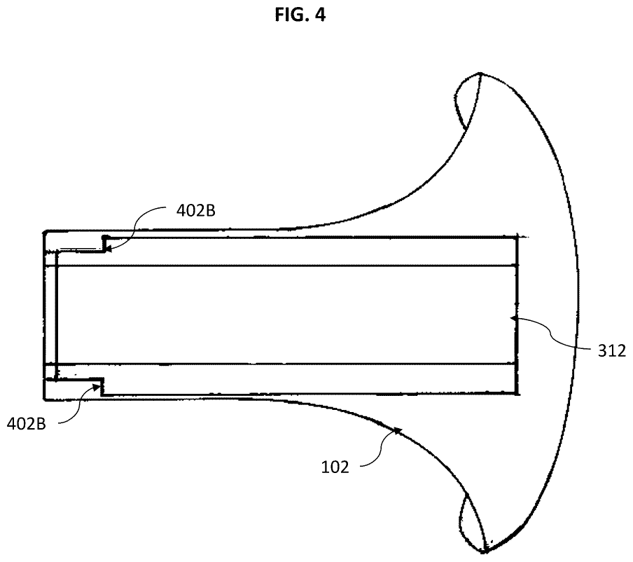

In some examples of the technology, it may be advantageous for the front portion 102 to remain connected to the rear portion 104 in use, and accordingly it can be beneficial for the hinged mechanism 106 to remain attached to the front 102 and rear portions 104 in use. Accordingly, FIG. 4 shows one example of how the channel 312 may be configured within the front portion 102 of the mask system 100. In the illustrated example the channel 312 comprises one or more stops 402A, 402B, which in use, engage with one or more protrusions 320A, 320B on the hinge mechanism 106 (see for example FIG. 3A). This provides a retention mechanism which retains the hinged mechanism 106 within the front portion 102, while allowing for sliding movement between the hinged mechanism 106, and the front portion 102.

Additionally, some examples of the technology also comprise a retention mechanism 322 configured to secure the front portion 102 at a substantially fixed position with respect to the rear portion 104. In the example of FIG. 3D, this retention mechanism 322 is provided by way of two complementary sections of hook-and-loop fastener 324A, 324B. The use of a hook-and-loop fastener should not be seen as limiting on the scope of the technology, and in other examples the retention mechanism may comprise any one or more of a clamp, buttons, domes, hooks, buckles, a latch, or any other suitable retention mechanism known to those skilled in the art.

In the illustrated examples, the retention mechanism 322 is shown as a pivoting flap 326 rotatably attached to the front portion of the mask system 100, however this should not be seen as limiting, and the retention mechanism need not be pivotally attached, or attached to the front portion. For example, the retention mechanism instead be attached to the rear portion, and releasably attach to the front portion in use.

### Examples

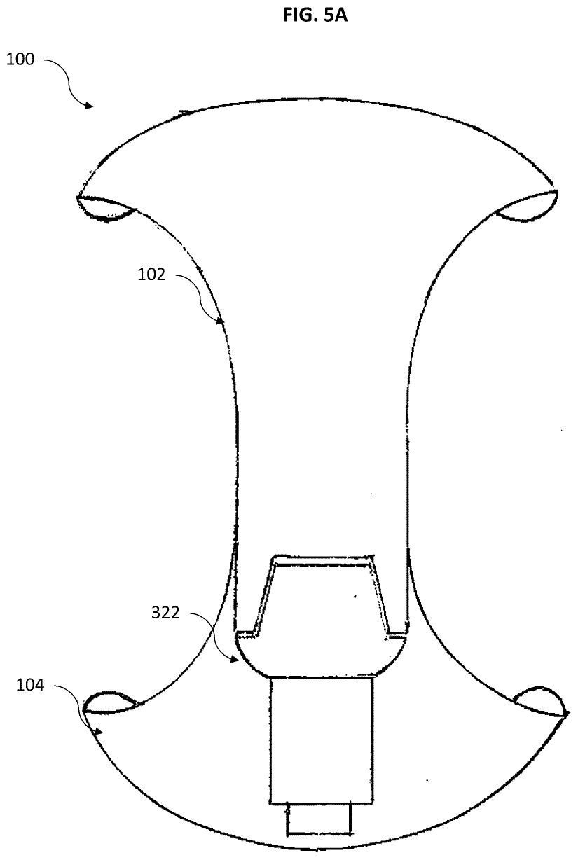

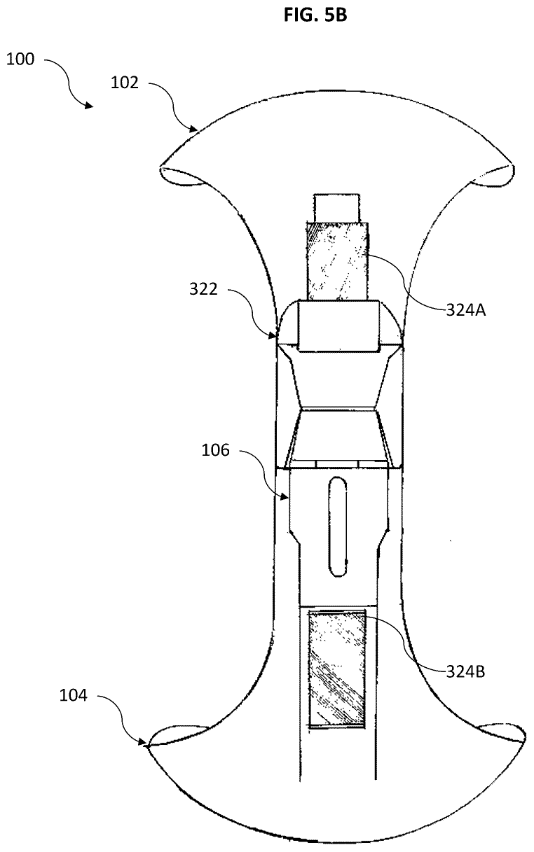

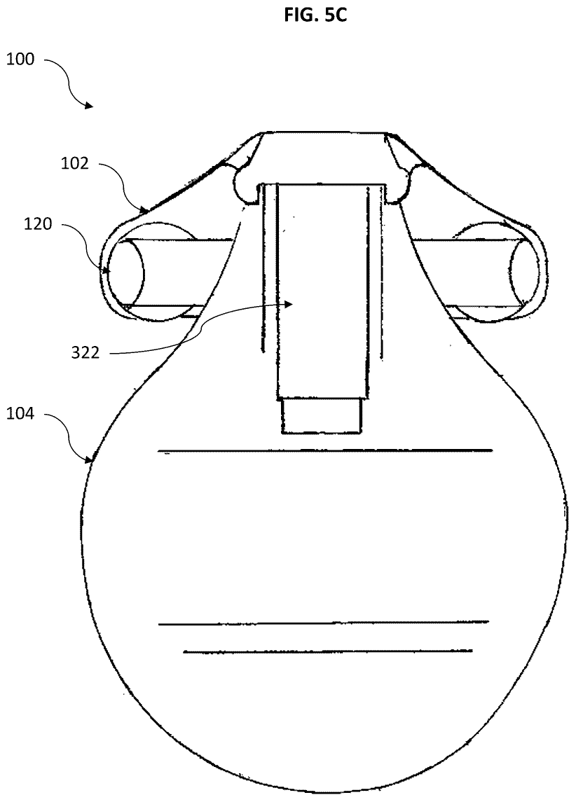

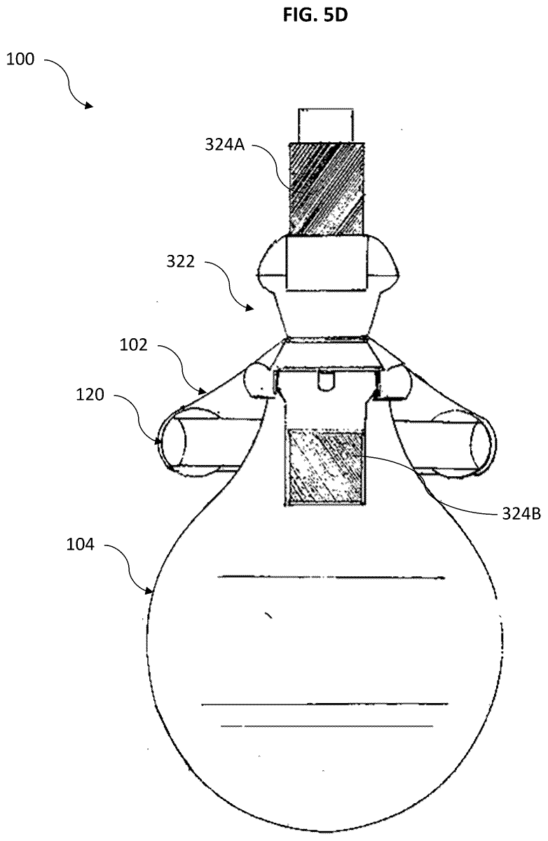

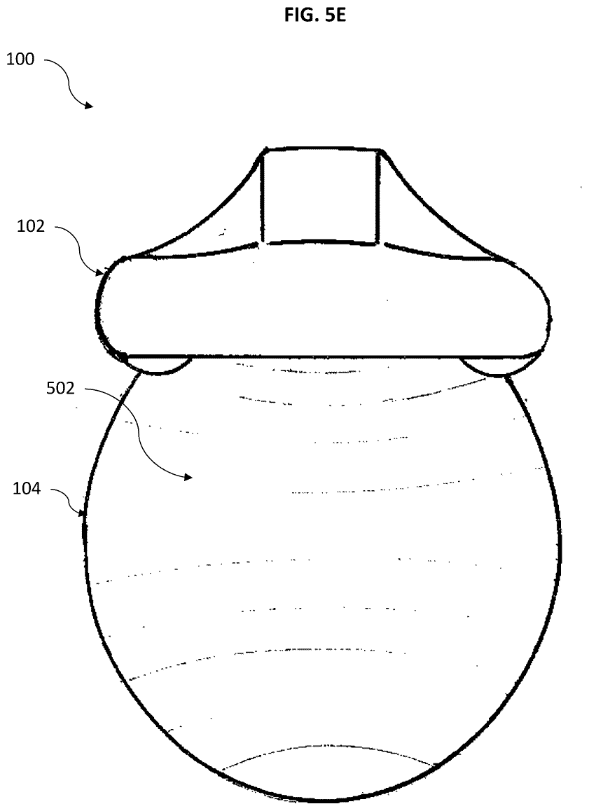

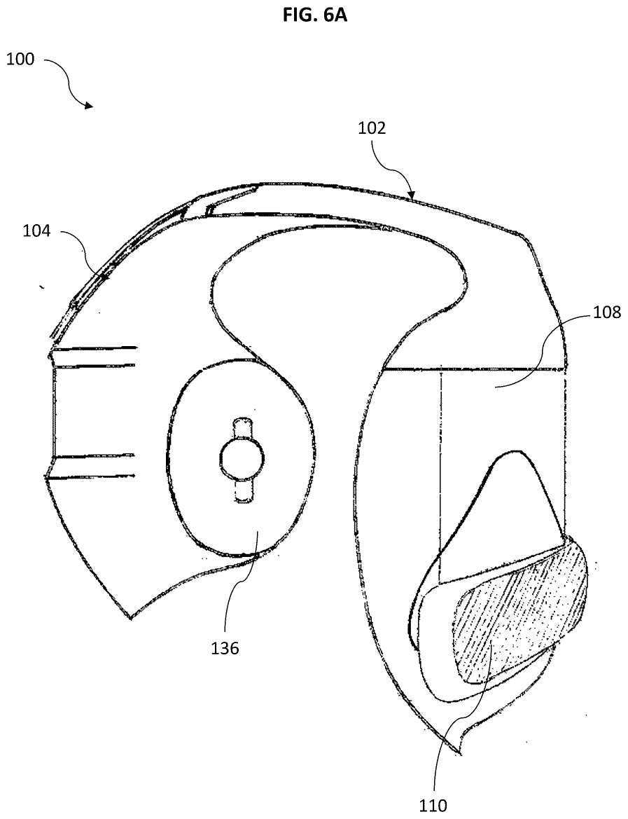

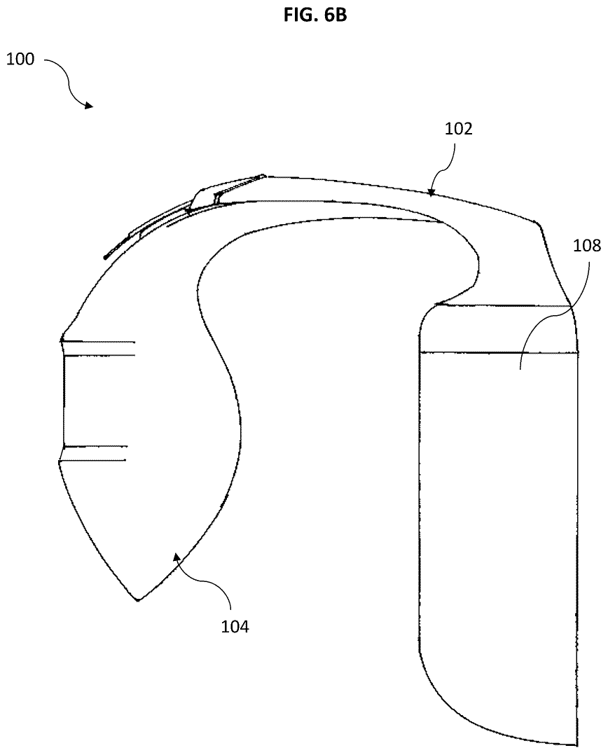

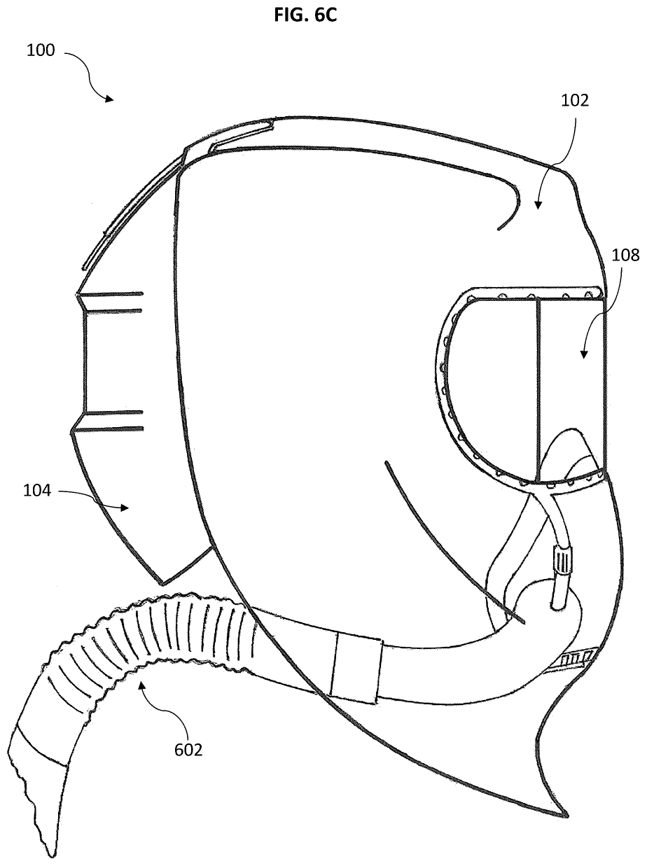

For completeness and to aid in the reader's understanding of the technology, additional views of the mask systems described herein are provided including:
FIG. 5A shows a top view of a mask system 100, wherein the retention mechanism 322 is currently in place, securing the front portion 102 to the rear portion 104.
FIG. 5B shows a top view of a mask system, wherein the retention mechanism 322 is disengaged, and not securing the front portion 102 to the rear portion 104. In this example the hinged mechanism 106 is currently withdrawn from the channel 312 such that pivotal movement of the front portion with respect to the rear portion is possible.
FIG. 5C shows a rear view of a mask system 100, wherein the retention mechanism 322 is currently in place, securing the front portion 102 to the rear portion 104. In this example the adjustment mechanism 120 is also shown in the forehead region of the mask system 100.
FIG. 5D shows a rear view of a mask system 100, wherein the retention mechanism 322 is currently disengaged, and not securing the front portion 102 to the rear portion 104. In this example the adjustment mechanism 120 is also shown in the forehead region of the mask system 100.
FIG. 5E shows a frontal view of a mask system 100 in which the resiliently deformable material 502 is shown in the crown, and/or nape regions of the rear portion 104.
FIG. 6A shows another example of a mask system comprising a visor 108, ear protection 136, and a breathing filter 110.
FIG. 6B shows another example of a mask system comprising a visor 108 in the form of a face shield.
FIG. 6C shows another example of a mask system comprising a visor 108, and an air conduit 602, which is connected to a source of breathable air in use connected to an internal face engaging seal 112 as described herein.

Accordingly, the present technology provides a mask system that comprises: a front portion configured to engage with the front of a user's head in use; a rear portion configured to engage with the rear of the user's head in use; and a hinge mechanism which connects the front portion to the rear portion, wherein in use the hinge mechanism is configured to move between a first position, in which the hinge mechanism allows for one of the rear portion and the front portion can be pivoted relative to the other of the rear portion and front portion, and a second position, in which the hinge mechanism does not allow one of the rear portion and the front portion to be pivoted relative to the other of the rear portion and front portion.

Alternatively, or additionally the present technology provides a mask comprising: a front portion configured to engage with the front of a user's head in use; and a rear portion configured to engage with the rear of the user's head in use. Wherein the one or more of the front portion and the rear portion comprise a hinge mechanism, configured to move within a channel 312 in the other of the front portion or the rear portion, to allow the front portion to slide with respect to the rear portion, and wherein the hinge mechanism comprises a joint 306, and when the joint 306 is withdrawn from the channel, the hinge mechanism is able to pivot around the joint 306 to allow the front portion to pivot relative to the rear portion or vice versa.

In another example of the technology, there is provided a hinge mechanism for use in a mask system, the hinge mechanism comprising, a first hinge component 302, a second hinge component 304 and a joint 306 connecting the first hinge component 302 to the second hinge component 304, such that the first hinge component 302 can pivot around the joint 306. Wherein the hinge mechanism is configured to attach to one or more of a front portion 102, or a rear portion 104 of a mask system 100, and the hinge mechanism 106 is configured to slide within a channel 312 in the other of the front portion 102 or the rear portion 104. In other words, the hinge mechanism 106 described herein provides an interconnection between the front portion 102 and the rear portion 104 which is slidably adjustable, and selectively allows the front portion to pivot with respect to the rear portion.

In yet a further example of the technology there is provided a method of putting on (donning) a mask system, comprising the steps of:
A) Slidably moving a front portion of a mask system relative to a rear portion of the mask system so as to remove a hinge mechanism from a channel;
B) Pivoting the front portion relative to the rear portion to increase the separation between the front portion and the rear portion;
C) Positioning the mask system over the head of the user;
D) Pivoting the front portion relative to the rear portion to decrease the separation between the front portion and the rear portion;
E) Sliding the front portion of the mask system relative to the rear portion of the mask system to as to insert the hinge mechanism into the channel; and
F) Optionally, attaching a securing mechanism between the front portion and the rear portion to prevent the front portion and rear portion sliding relative to each other.

In yet a further example of the technology there is provided a method of removing (doffing) a mask system, comprising performing the aforementioned steps in reverse, in other words, to:
G) Optionally, remove a securing mechanism between the front portion and the rear portion to allow the front portion and rear portion to slide relative to each other.
H) Slide the front portion of the mask system relative to the rear portion of the mask system to as to remove the hinge mechanism from the channel;
I) Pivot the front portion relative to the rear portion to decrease the separation between the front portion and the rear portion;
J) Remove the mask system from the head of the user;
K) Optionally, pivoting the front portion relative to the rear portion to decrease the separation between the front portion and the rear portion; and
L) Optionally, sliding the front portion of the mask system relative to the rear portion of the mask system so as to remove the hinge mechanism from the channel.

### Sliding Guides

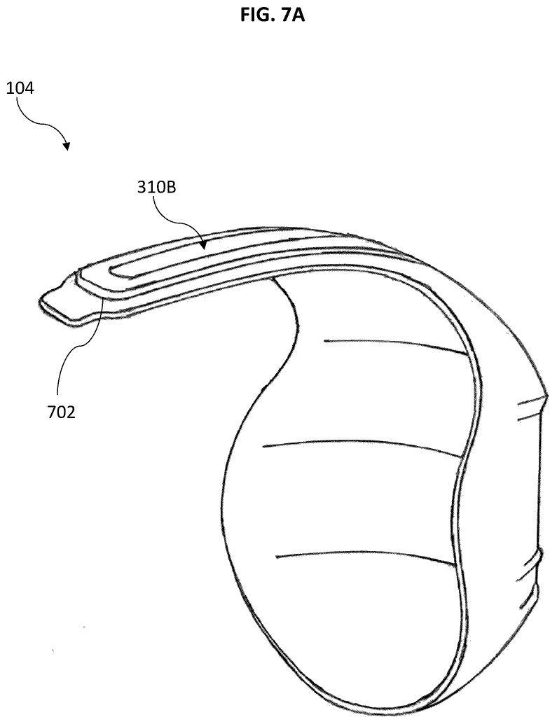

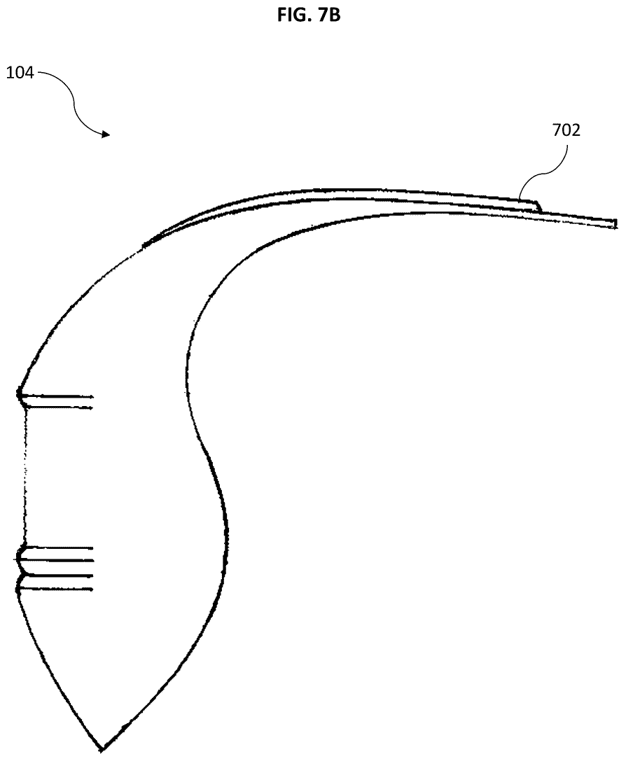

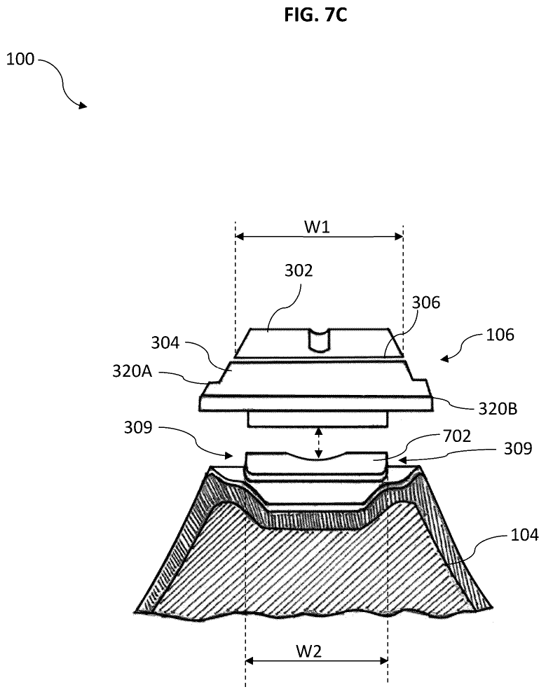

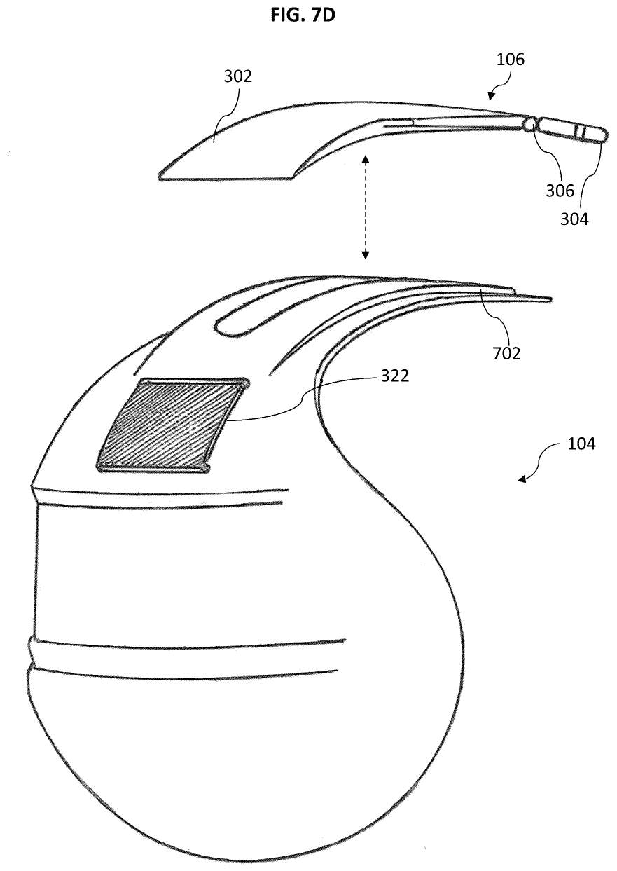

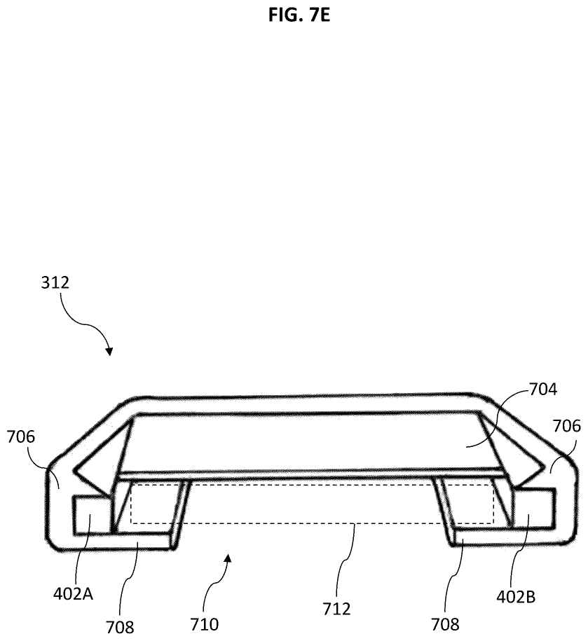

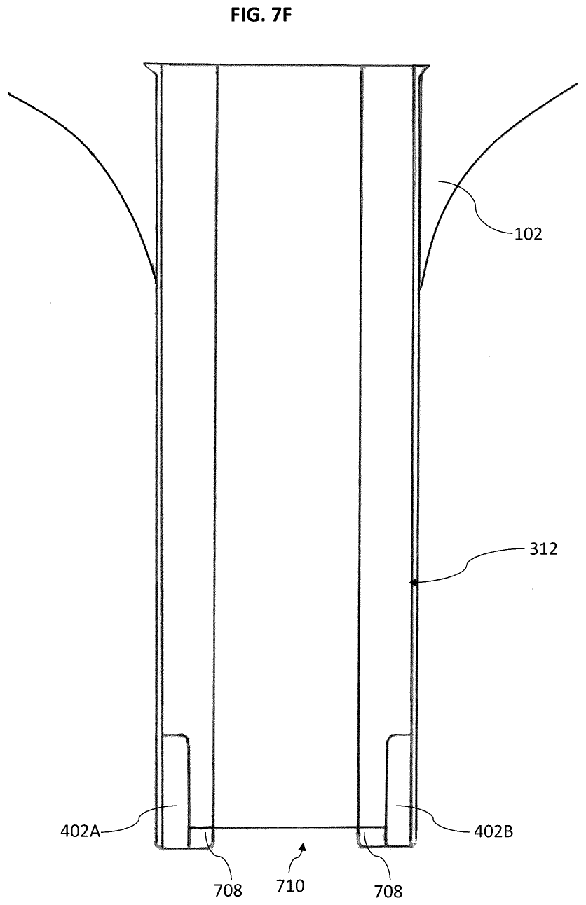

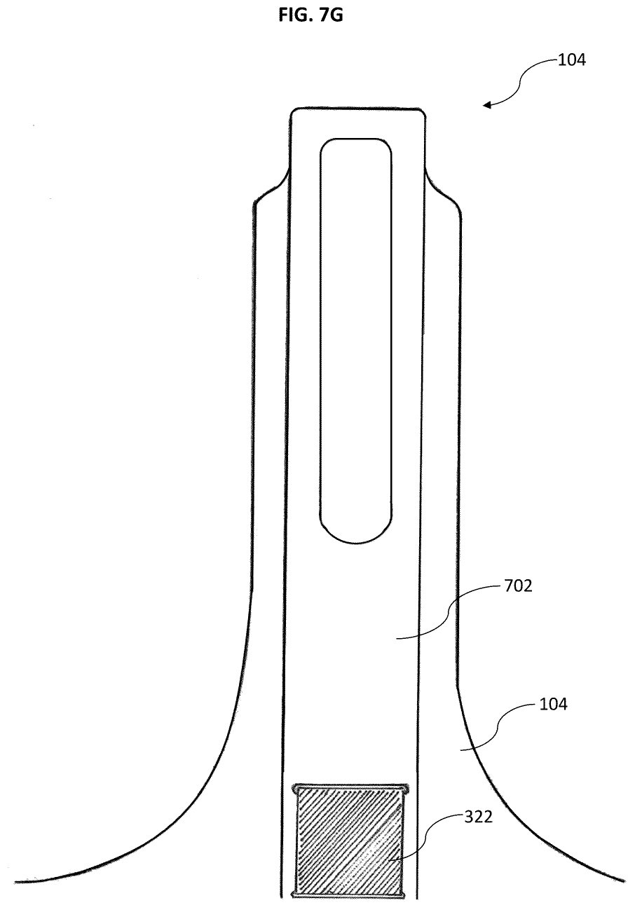

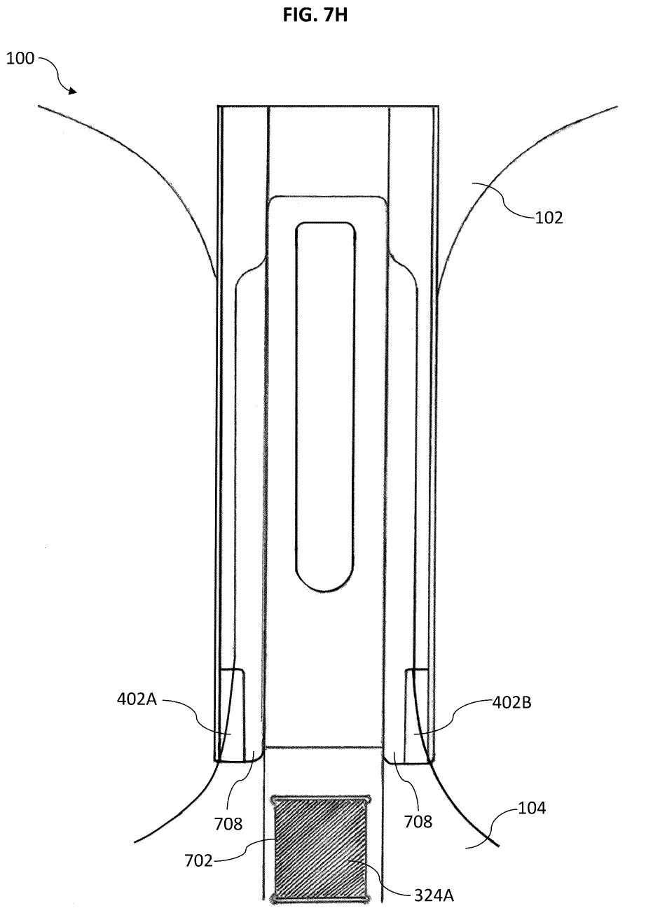

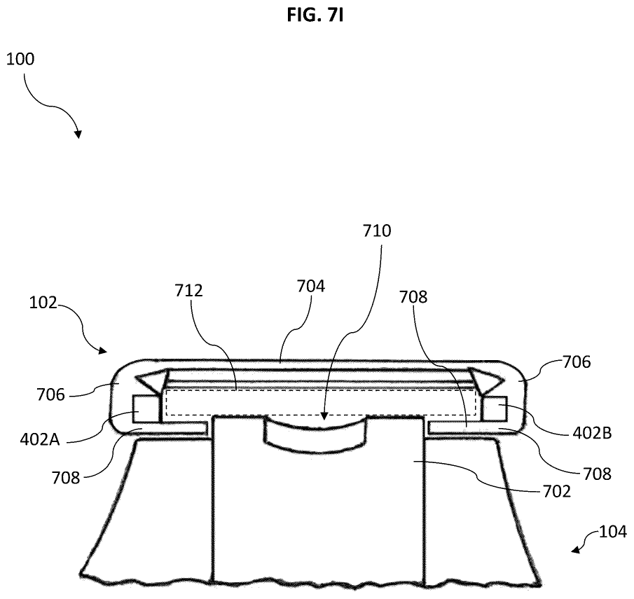

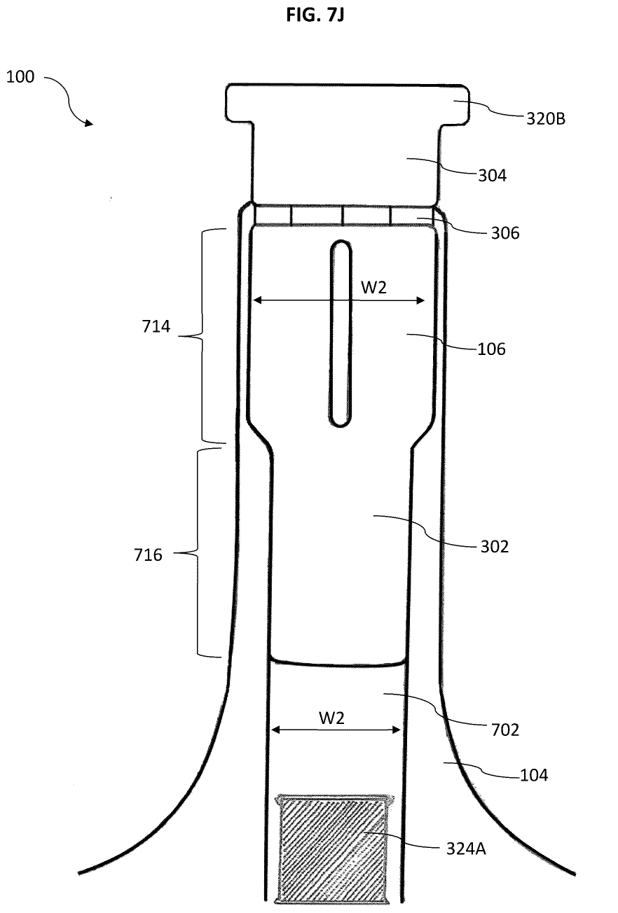

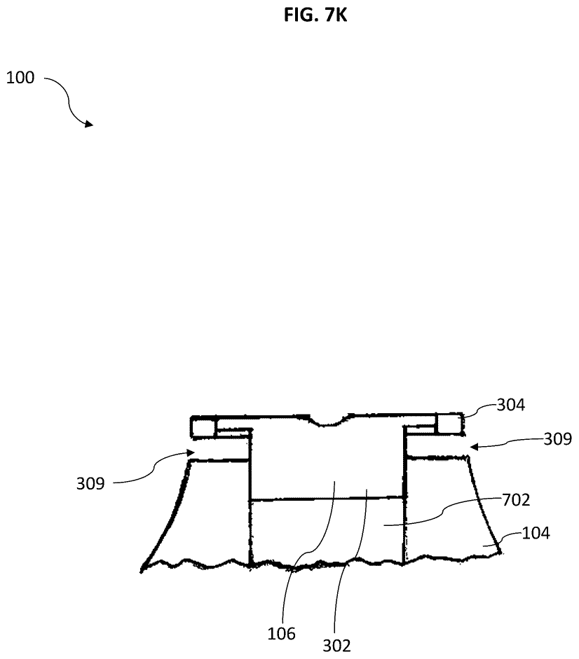

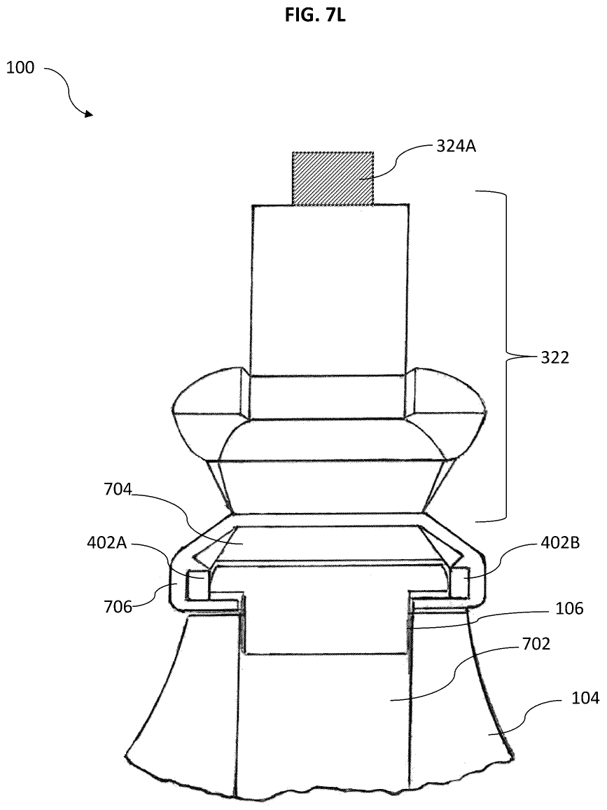

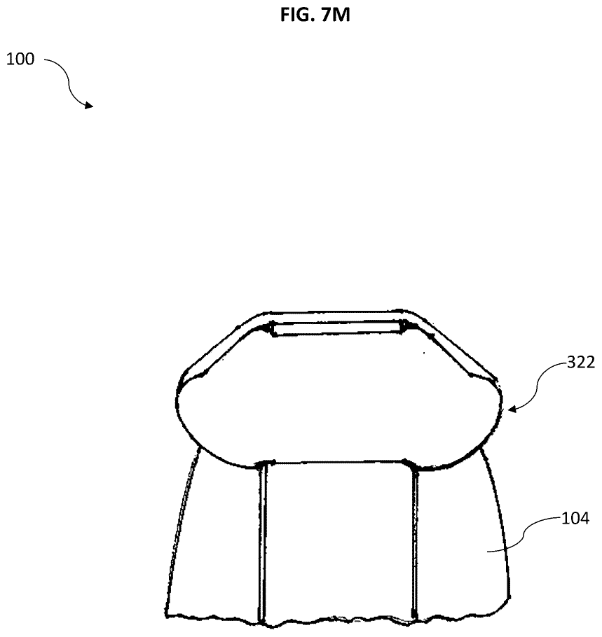

FIGS. 7A to 7M, show additional views of the components of a mask system 100 in accordance with the present technology. With reference to FIGS. 7A and 7B, some examples of the present technology may include a rear portion 104, which has a raised section or ridge 702. The raised section 702 may provide a number of advantages with the present technology including, acting as a guide which defines the axis of movement of the rear portion 104 with respect to the front portion 102, as well as providing a track 309 beneath the hinge mechanism 106, when the hinge mechanism is mounted to, or otherwise attached to the section portion 702.

Also shown in FIG. 7A is the complementary structure 310B, which in use may engage with the guide 310 in the hinge mechanism 106 to thereby align and/or attach the hinge mechanism 106 to the rear portion 104.

The use of a raised section however should not be seen as limiting on the technology. For example, the hinge mechanism 106 may be provided with a rebated section having a reduced width in comparison with the first hinge component 302, such that when the hinge mechanism 106 is attached to the rear portion 104, the rebated section raises the first hinge component 302 above the surface of the rear portion, to thereby create a track 309 under the sides of the rear portion.

FIGS. 7C and 7D show examples of how the hinge mechanism 106 may be positioned relative to the raised portion 702. It should be appreciated that these are exploded views, and in use, the hinge mechanism may be mounted to the raised section 702. As shown, it can be seen that the hinged mechanism 106, may have a width W1, which is greater than a width of the raised portion W2, so that the edges of the first hinge component 302 overhang the raised section 702 to create a track located in the area indicated by line 309.

FIG. 7E shows an example of a channel 312 as discussed in relation to the previous embodiments of the technology. The channel comprises a top surface 704, side walls 706, which extend downwardly from the top surface, substantially perpendicular to the top surface 704, and ledges 708 which extend inwardly from the side walls 706 in a direction which is substantially parallel to the top surface 704. In use a gap 710 between the ledges 708 is configured to receive the raised portion 702, as illustrated in FIG. 7I while the channel region 712 which is formed above the ledges 708 is configured to receive the hinge mechanism 106, as illustrated in FIG. 7L. Stops 402A, and 402B are provided which in use prevent the hinge mechanism 106 from being completely withdrawn from the channel 312, as the stops 402A, 402B on the channel engage with the protrusions 320A, 320B as described herein.

In some examples, the channel 312 is formed in or otherwise attached to the front portion 102 of the mask system 100. For example, in the illustrated example, the channel 312 may be provided by a component which is attached to the front portion of the mask system 100, or otherwise integral to the mask system 100. For example, the top surface 704 of the channel 312 may be provided by an underside surface of the front portion 102. Similarly, the sidewalls 706, and ledges 708 may either be formed as part of or otherwise attached to the front portion 102.

FIG. 7F shows a top-down view of a front portion 102 where the channel 312 is illustrated. It should be appreciated that in some examples of the technology, the channel 312 is provided to an underside of the front portion 102 and would not typically be visible from the top. Nevertheless, the illustration of FIG. 7F shows an outline of the channel to aid in the understanding of the technology.

FIG. 7G, shows a corresponding top-down view of a rear portion 104, with the hinge mechanism 106 removed. As shown, the rear portion 104 comprises a raised section 702, and retention mechanism 322 in the form of a hook and loop fastener.

FIG. 7H, shows a top down view of the rear portion 104 inserted into the channel 312 in the front portion 102. In this view, together with the corresponding rear view of FIG. 7I, it can be seen that the raised portion 702, is configured to travel through the gap 710, such sides of the raised portion are guided by the ledges 708. The length of the raised portion 702, together with the corresponding channel 312, can be beneficial in ensuring that the rear portion 104 cannot twist or yaw within the front portion 102 or vice-versa. As in the previous examples, the hinge mechanism 106 is not shown in FIG. 7I for sake of clarity, however it should be appreciated that, in use, the hinge mechanism 106 is configured to extend through the channel region 712, to thereby prevent the rear portion 104 from coming away from the front portion 102, while allowing the rear portion 104 and front portion 102 to slide with respect to each other to control how much of the hinge mechanism is positioned within the channel region 712. When at least part of the first hinge component 302 is positioned within the channel 312, the front portion 102 is substantially rotationally fixed relative to the rear portion 104, and wherein the depth of the first hinge component within the channel defines the anterior-posterior sizing of the mask system 100. When the first hinge component 302 is removed from the channel 312 (for example when the protrusions 320A, 302B engage with the stops 420A, 420B) the front portion 102 is free to pivot with respect to the rear portion 104.

FIG. 7J shows a top-down view of a rear portion 104, which includes a hinge mechanism 106 positioned on a raised portion 702 as described herein. In this example, the hinge mechanism 106 includes a wide portion 714, with a width W2, which overhangs the raised portion 702 to create the track 309, and a narrow portion 716, which in the illustrated example has a width which substantially corresponds to the width of the raised portion W2, although this should not be seen as limiting on the scope of the technology, and any suitable width may be used including variable widths, and constant widths.

FIG. 7K shows a rear view of the hinge mechanism 106 situated on top of the rear portion 104, so as to form the tracks 309 as described herein.

FIGS. 7L and 7M show respective rear perspective views of the mask system with the hinge mechanism 706 engaged in the channel 312 to thereby join the front portion 102 to the rear portion 104. In the example of FIG. 7L the retention mechanism 322 is shown in an open position which allows for relative adjustment between the front 102 and rear portions 104 by sliding the hinge mechanism 106 along the channel, while FIG. 7M show a corresponding view with the retention mechanism 322 in a closed position which substantially prevents movement of the front portion 102 with respect to the rear portion 104.

### Other Matters

Throughout the present specification reference to the workshop mask systems should be understood to include any mask system, including helmets which are used in workshop applications such as fabrication, welding, grinding, painting, casting, moulding, glassblowing, sanding, galvanising, or any other suitable activity which may require protection, such as a physical barrier, visual barrier, audio barrier, air filtering or an independent air supply. For example, the workshop mask may be configured to protect the user against any one or more of: heat, dust, cuts, particles, sparks, chemicals, fumes etc.

The illustrated embodiments of the disclosure will be best understood by reference to the figures. The foregoing description is intended only by way of example and simply illustrates certain selected exemplary embodiments of the disclosure.

The entire disclosures of all applications, patents and publications cited above and below, if any, are herein incorporated by reference. Reference to any prior art in this specification is not, and should not be taken as, an acknowledgement or any form of suggestion that that prior art forms part of the common general knowledge in the field of endeavour in any country in the world.

The invention(s) of the present disclosure may also be said broadly to consist in the parts, elements and features referred to or indicated in the specification of the application, individually or collectively, in any or all combinations of two or more of said parts, elements or features. Where in the foregoing description reference has been made to integers or components having known equivalents thereof, those integers are herein incorporated as if individually set forth.

Furthermore, the described features, structures, or characteristics may be combined in any suitable manner in at least one embodiment. In the foregoing description, numerous specific details are provided to give a thorough understanding of the exemplary embodiments. One skilled in the relevant art may well recognize, however, that embodiments of the disclosure can be practiced without at least one of the specific details thereof, or can be practiced with other methods, components, materials, et cetera. In other instances, well-known structures, materials, or operations are not shown or described in detail to avoid obscuring aspects of the invention.

Throughout this specification, the word “comprise” or “include”, or variations thereof such as “comprises”, “includes”, “comprising” or “including” will be understood to imply the inclusion of a stated element, integer or step, or group of elements integers or steps, but not the exclusion of any other element, integer or step, or group of elements, integers or steps, that is to say, in the sense of “including, but not limited to”.

Aspects of the present disclosure have been described by way of example only and it should be appreciated that modifications and additions may be made thereto without departing from the scope thereof.

1. A mask comprising:
a front portion configured to engage with the a front of a user's head in use;
a rear portion configured to engage with the a rear of the user's head in use;
a hinge mechanism which connects the front portion to the rear portion; and
wherein, in use, the hinge mechanism is configured to move between a first position, in which the hinge mechanism allows for pivotal movement of one of the front portion and rear portion relative to the other of the front portion and rear portion, and a second position, in which the hinge mechanism does not allow for pivotal movement of one of the front portion and rear portion relative to the other of the front portion and rear portion.

2. The mask as claimed in claim 1, wherein the front portion comprises any one or more of: a visor, a breathing filter, a face engaging seal, a face shield, an air filter, air vents, and/or an air conduit.

3. The mask as claimed in claim 1, wherein the front portion comprises side portions which extend in a substantially posterior direction from the front portion.

4. The mask as claimed in claim 1, wherein the front portion and the rear portion together provide a mask which substantially encloses the head of the user.

5. The mask as claimed in claim 1, wherein the front portion comprises a forehead contacting portion which is adjustably mounted to the front portion via an adjusting means.

6. The mask as claimed in claim 1, further comprising a crown or nape engaging portion configured to engage with the crown, and or nape of the user in use, wherein the crown or nape engaging portion is adjustably mounted to the rear portion by an adjusting means.

7. The mask as claimed in claim 1, wherein the hinge mechanism comprises a first hinge component and a second hinge component, the first hinge component being rotatably connected to the second hinge component.

8. The mask as claimed in claim 7, wherein the first hinge component is slidably engaged with the front portion, and the second hinge component is fixed to the rear portion.

9. The mask as claimed in claim 1, wherein the hinge mechanism is configured to slide within a channel in the front portion.

10. The mask as claimed in claim 9, wherein the hinge mechanism comprises a first hinge component and a second hinge component, the first hinge component being rotatably connected to the second hinge component and wherein the first hinge component is slidably engaged with the front portion and the second hinge component is fixed to the rear portion; and
wherein when the second hinge component is external to the channel, the hinge mechanism allows for pivotal movement of one of the front portion and rear portion relative to the other of the front portion and rear portion, and when the second hinge component is positioned within the channel, the hinge mechanism does not allow for pivotal movement of one of the front portion and rear portion relative to the other of the front portion and rear portion.

11. The mask as claimed in claim 9, wherein the hinge mechanism and channel have a complementary arcuate profile when viewed from the side.

12. The mask as claimed in claim 9, wherein the hinge mechanism comprises a guide which is configured to engage with a complementary feature in the channel, so as to guide the hinge mechanism within the channel while minimising any yawing motion.

13. The mask as claimed in claim 9, further comprising a retention mechanism configured to secure the hinge mechanism at a selected location within the channel.

14. The mask as claimed in claim 1, wherein the hinge mechanism comprises a flange, which provides a channel between the hinge mechanism and the rear portion, and wherein in use the front portion is configured to slide along the channel between the flange and the rear portion.

15. A mask system comprising:
a front portion configured to engage with a front of a user's head in use;
a rear portion configured to engage with a rear of the user's head in use;
wherein one of the front portion and the rear portion comprise a hinge mechanism, configured to move within a channel provided to an other of the front portion and the rear portion, to allow the front portion to slide with respect to the rear portion; and
wherein the hinge mechanism comprises a joint, and when the joint is withdrawn from the channel, the hinge mechanism is able to pivot around the joint to allow the one of the front portion and rear portion to pivot relative to the other of the front portion and rear portion.

16. A hinge mechanism configured for use in a mask system, the hinge mechanism comprising:
a first hinge component, and
a second hinge component,
wherein the first hinge component is pivotally connected to the second hinge component, and wherein, in use first hinge component is configured to attach to a first component of a mask system, and the second hinge component is configured to attach to a second component of a mask system such that the second hinge component can slide within a channel in the second component,
wherein the hinge mechanism is configured to slide within the channel, between a first position in which the first hinge component is external to the channel, and a second position in which the first hinge component is positioned within the channel, and
wherein in the first position the hinge mechanism allows the first component to pivot relative to the second component, and in the second position the hinge mechanism prevents the first component from pivoting relative to the second component.

17. The hinge mechanism as claimed in claim 16, wherein the hinge mechanism has a curved profile when viewed from the side.

18. The hinge mechanism as claimed in claim 16, further comprising a guide configured to guide the hinge mechanism within the channel while minimising any yawing motion of the hinge mechanism within the channel.

19. A method of using a mask as claimed in claim 1, comprising the steps of:
positioning the mask on the head of a user;
pivoting the front portion of the mask relative to the rear portion of the mask, so as to align the hinge mechanism with a channel;
sliding the hinge mechanism into the channel until the front portion engages with a front of the user's head and the rear portion engages with the rear of the user's head.

20. The method of claim 19, further comprising the step of: attaching a securing mechanism between the front portion and the rear portion to prevent the front portion and rear portion sliding relative to each other.
